(19) United States
(12) Patent Application Publication
(10) Pub. No.: US 2011/0184498 A1
(43) Pub. Date: Jul. 28, 2011

(54) Interactive vision therapy board

(76) Inventor: Robin Lynn Donley, San Diego, CA (US)

(21) Appl. No.: 12/925,163

(22) Filed: Oct. 13, 2010

Related U.S. Application Data
(60) Provisional application No. 61/278,819, filed on Oct. 13, 2009.

(51) Int. Cl. A61N 5/06

(52) U.S. Cl. 607/88

(57) ABSTRACT

A light board machine useful for visual training and therapy purposes includes a planar backboard having an array of light locations having two colored lights (red and green), a microprocessor controller for controlling the operation, sequencing and timing of the lights, and a user-feedback device, such as a touch-activated panel, which cooperate to provide visual training and therapy functions. The present invention includes the option to use red (lights) only, green only or red and green in a random order. The present invention also includes letters and numbers behind some of the lights (e.g. 80 of the 120 lights), and has a sequential pattern option of 30, 60, 90 or 120 lights. This invention is compatible with the use of red/green glasses to accommodate training of binocularity and depth. This invention is used in a therapy setting and will helps to enhance eye hand coordination, response/reaction time, peripheral vision/awareness, localization, awareness of space and depth perception, eye tracking (saccades and occulomotor), sequential movement and processing, as well as a cognitive loading component that therapists use in a variety of ways for individual patients and specific therapy modalities.

### RELATED APPLICATIONS

This application claims the benefit of priority to U.S. Provisional Application Ser. No. 61/278,819 filed Oct. 13, 2009, by the present inventor for an invention entitled “Interactive Vision Therapy Board”, currently co-pending.

### FIELD OF THE INVENTION

The present invention relates general to vision therapy devices. The present invention is more particularly, though not exclusively, useful as a visual therapy training device.

### BACKGROUND OF THE INVENTION

The present invention relates to visual training. In order to fully appreciate the functions and benefits of the present invention, it is necessary to discuss what is, visual training, and how it can be enhanced.

Nearly all humans are born with the potential for good eyesight, but vision—the ability to identify, interpret and understand what is seen—is learned and developed, starting at birth. In learning to walk, a child begins by creeping, crawling, standing, walking with assistance, and finally, walking unaided. A similar process from gross to fine motor control takes place in the development of vision. One visual skill builds on another, step-by-step as we grow. But many people miss a step, or do not complete a step, or must begin to perform school or other visually demanding tasks before an acceptable foundation of basic visual skill is in place.

Science indicates that we do not “see” with our eyes or our brain; rather, vision is the reception and processing of visual information by the total person. Since two-thirds of all information we receive is visual, it becomes clear that efficient visual skills are a critical part of learning, working and even recreation. Athletes, for example, use visual training for improved performance in their sport.

Developing visual skills includes learning to use both eyes together effectively. Having both eyes move, align, fixate and focus as a team enhances your ability to interpret and understand the potential visual information that is available to you.

Intelligent persons who are very highly motivated can be good achievers, even with very poor visual skills and abilities, but at untold cost in wasted energy and unnecessary effort and stress. For those who are less motivated, even one or two deficient visual skills can produce enough stress and frustration to create a non-achiever

The visual skills which can be developed and enhanced through visual training include: Tracking—The ability to follow a moving object smoothly and accurately with both eyes, such as a ball in flight or moving vehicles in traffic; Fixation—The ability to quickly and accurately locate and inspect with both eyes a series of stationary objects, one after another, such as moving from word to word while reading; Focus Change—The ability to judge relative distances of objects and to see and move accurately in three-dimensional space, such as when hitting a ball or parking a car; Peripheral Vision—The ability to monitor and interpret what is happening around you while you are attending to a specific central visual task; the ability to use visual information perceived from over a large area; Binocularity—The ability to use both eyes together, smoothly, equally, simultaneously and accurately; Maintaining attention—The ability to keep doing any particular skill or activity with ease and with interfering with the performance of other skills; Near Vision Acuity—The ability to clearly see, inspect, identify and understand objects at near distances, within arm's length; Distance Acuity—The ability to clearly see, inspect, identify and understand objects at a distance. People with 20/20 distance sight still may have visual problems; and Visualization—The ability to form mental images in your “mind's eye,” retain or store them for future recall, or for synthesis into new mental images beyond your current or past direct experiences.

Optometric visual training, sometimes called vision therapy or VT, is that part of optometric care devoted to developing, improving and enhancing people's visual performance. Vision therapy can benefit people of all ages. Optometrists have developed and used visual training for several decades to prevent vision and eye problems from developing, develop the visual skills needed to achieve more effectively at school, work or play, enhance functioning on tasks demanding sustained visual effort, and remediate or compensate for vision and eye problems which have already developed.

Visual training also has proven to be a remarkably effective tool in helping people with learning-related visual problems. Many problems in learning to read and write are made worse by poorly developed visual skills. Dozens of experimental programs involving thousands of children and adults demonstrate that when visual skills are enhanced through visual training, learning is easier, reading levels rise, and in some cases, IQ scores have increased. Building visual skills also increases the ability to visualize, conceptualize and to create.

Vision therapy (visual training, vision training) is an individualized supervised treatment program designed to correct visual-motor and/or perceptual-cognitive deficiencies which have various causes, such as inadequate sensorimotor development, trauma to the nervous system (i.e., birth injury, brain trauma, closed head trauma, etc.), stress, in some cases, contributing hereditary factors (i.e., crossed-eyes).

Typical vision therapy sessions may include procedures designed to enhance the brain's ability to control eye alignment, eye movements, focusing abilities, and eye teamwork (binocular vision). Visual-motor skills and endurance are developed through the use of specialized computer and optical devices, including therapeutic lenses, prisms and filters. During the final stages of therapy, the patient's newly acquired visual skills are reinforced and made automatic through repetition and by integration with motor and cognitive skills.

For over 35 years the Wayne Saccadic Fixator (“Circle of Lights”) has been the developmental optometric profession's standard for testing, evaluating, and developing accurate and rapid eye-hand coordination, spatial integration, and reaction times. Athletic trainers, developmental vision optometrists, and rehabilitation therapists have acclaimed the Wayne Saccadic Fixator for its ability to motivate users. The Wayne Saccadic Fixator has been used for sports vision training by college, professional, and Olympic teams worldwide, including the 1980 “Miracle on Ice” US Olympic hockey team.

The Wayne Saccadic Fixator is a wall-mounted instrument with a touch-sensitive membrane panel containing 33 LED lights arranged in three concentric circles with one light at the center. The user responds to the appearance of a light by pressing the membrane button surrounding it.

The device presents a number of activities based on several functions, including: self-pacing—the unit presents a light and waits for the user to press it; auto-pacing—the unit presents lights at a preset speed; only lights pressed within the time limit count; auto-pacing with speed tracking—the presentation speed increases or decreases according to the user's performance; visual memory—the unit presents a sequence of random lights of increasing length, which the user has to repeat; and reaction/anticipation timing—using the Eye-hand reaction/release timing.

The Wayne Saccadic Fixator provides a repetitive sequence of lights for training purposes. However, for a repeat patient, this sequence of lights becomes too familiar and therefore a falsely high score can be achieved through memorization of the sequence and not improvement of eye-hand coordination.

Despite the understanding of the importance of visual training and therapy, there has been little advancement in devices available to that industry to accomplish these training and therapy tasks. As a result, the currently available devices fall short in optimizing the training regimens.

In light of the above, it would be advantageous to provide a visual training device that overcomes many of the current challenges, and provides a training and therapy device which provides visual trainers and therapists with more options for patient-specific therapies.

The present invention differs from previous art by utilizing two colored lights (red and green), and by providing the option to use red (lights) only, green only or red and green in a random order. The present invention also differs in that it includes letters and numbers behind some of the lights (e.g. 80 of the 120 lights), and has a sequential pattern option of 30, 60, 90 or 120 lights. This invention will be compatible with the use of red/green glasses.

### SUMMARY OF THE INVENTION

The present invention is a light board machine useful for visual training and therapy purposes. The present invention includes a planar backboard having an array of light locations having two colored lights (red and green), a microprocessor controller for controlling the operation, sequencing and timing of the lights, and a user-feedback device, such as a touch-activated panel, which cooperate to provide visual training and therapy functions.

The present invention includes the option to use red (lights) only, green only or red and green in a random order. The present invention also includes letters and numbers behind some of the lights (e.g. 80 of the 120 lights), and has a sequential pattern option of 30, 60, 90 or 120 lights. This invention is compatible with the use of red/green glasses to accommodate training of binocularity and depth.

This invention is used in a therapy setting and will helps to enhance eye hand coordination, response/reaction time, peripheral vision/awareness, localization, awareness of space and depth perception, eye tracking (saccades and occulomotor), sequential movement and processing. With the letters and numbers that are randomly placed on the lights this invention will have a cognitive loading component to it that therapist will be able to use in a variety of ways which can be customized for individual patients and specific therapy modalities.

Behavioral Optometry, Occupational Therapy, Physical Therapy, Rehabilitation Therapy, Speech and Language Therapy, Sports therapy will all be able to use the present invention in a therapy setting with their patients. Patients with brain injury's, acquired and traumatic, stroke patients, autism, patients with a variety of visual dysfunctions (strabismus, amblyopia, binocular dysfunction, dipolypia, monocular vision, partial visual field losses to name a few), will all benefit in a therapy setting.

The present invention provides benefits to patients including but not limited to the following categories: behavioral optometry; speech and language pathology; physical therapy; double vision; autism; brain Injury; speech therapy; and sports therapy.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a system level diagram of the interactive vision therapy board of the present invention showing a display panel containing a number of lights spaced apart on the panel, a fixator light positioned centrally, and a controller and remote computer attached and giving operational instructions to the display panel;

FIG. 2 is a front plan view of the display portion of the interactive vision therapy board of the present invention showing the black background and the placement of lights around the surface of the panel;

FIG. 3 is a black and white image showing the location of the lights in the panel, and depicts a number of zones that can be separately controlled by the controller;

FIG. 4 is a photographic example of the panel portion of the interactive vision therapy board of the present invention showing the selective illumination of lights on the panel, and even the simultaneous illumination of multiple lights;

FIG. 5 is a photographic example of the panel portion of the interactive vision therapy board of the present invention showing an alternative selection of illuminated lights;

FIG. 6 is a photographic example of the front of the panel portion of the interactive vision therapy board of the present invention showing the placement of lights across the panel;

FIG. 7 is a graphical representation of the panel portion of the interactive vision therapy board of the present invention showing the lights having letters, numbers, and neither letters or numbers;

FIG. 8 is an exemplary drawing of a switch member of the interactive vision therapy board of the present invention showing a circular touch pad and having an electronic connection tail for establishing electrical contact when the switch member is pressed;

FIG. 9 is an exemplary cross-sectional drawing of the panel portion of the interactive vision therapy board of the present invention showing the various layers comprising the display panel, and the deflection of the graphic overlay by depression of the panel by a finger, and the illumination pattern through the display;

FIG. 10 is an exemplary control panel of the interactive vision therapy board of the present invention showing user interface controls, including START, STOP, TIME, SCORE, LATE, SPEED, FIXATOR, AREA, MODE and COLOR;

FIG. 11 is a perspective view of the mounting system and rack of the interactive vision therapy board of the present invention, showing a wall mountable rack, and a slidable mounting support panel frame that is slidable upwards and downwards to all heights within the vertical supports;

FIG. 12 is a front plan view of the mounting system and rack of the interactive vision therapy board of the present invention showing the vertical movement of the support panel frame within the vertical supports;

FIG. 13 is an exemplary sequence of lights shown on the panel of the interactive vision therapy board of the present invention, and depicting a sequence which traverses the display panel;

FIG. 14 is a flow chart diagram showing the Random Time Trials operation of the interactive vision therapy board of the present invention;

FIG. 15 is a flow chart diagram showing the Random Time Trails with Color operation of the interactive vision therapy board of the present invention;

FIG. 16 is a flow chart diagram showing the Sequential Time with Speed≠0 operation of the interactive vision therapy board of the present invention;

FIG. 17 is a flow chart diagram showing the Fixator Active with Speed≠0 operation of the interactive vision therapy board of the present invention;

FIG. 18 is a front perspective view of the mounting system of the present invention showing a wall-mounted frame with a central slide frame that slides up and down within the wall mounted frame and is used to secure the display panel to the wall for adjustment and use;

FIG. 19 is a perspective view of the central slide frame showing an exploded brake assembly which is used to facilitate the vertical positioning of the slide within the wall mounted frame, and then by actuating the brake, locking the slide within the wall mounted frame for use;

FIG. 20 is an enlarged view of the brake assembly as it mounts to the corner of the central slide frame;

FIG. 21 is an enlarged view of the brake assembly with a top cover removed showing the cam as it is positioned between two brake pad assemblies that eject outwards to strike the interior of the wall mounted frame rail to secure the central slide frame in place;

FIG. 22 is an exploded view of the brake assembly shown in FIG. 21, and showing the insertion of the brake pads within a housing that is forced outwards when the central cam is rotated;

FIG. 23 is an exploded view of the pulley system of the present invention used to support the central slide frame within the wall mounted frame and balance the weight with a counterweight to facilitate the raising and lowering of the central slide frame and attached display panel;

FIG. 24 is a view of the wall mounted frame and showing the locking lever attached to the end of the cam rod that passes through the cam within the brake assembly so that the locking lever can be rotated 90 degrees to remove the forces from brake assembly in the central slide frame, the frame can be raised and lowered, and the locking lever can be returned to its position rotating 90 degrees to rotate the cam within the brake assembly to lock the central slide frame in place;

FIG. 25 is a view of the present invention showing the central frame and an associated brake assembly as positioned on cam rod and slidable up and down;

FIG. 26 is an enlarged view of the brake assembly as it passes over cam rode and showing the counter weight;

FIG. 27 is a view of the pulley orientation and positioning for use of the counterweight to balance the weight of the display panel; and

FIG. 28 is a detail view of the locking arm as shown to rotate the cam rod to lock the brake assemblies.

### DETAILED DESCRIPTION OF THE INVENTION

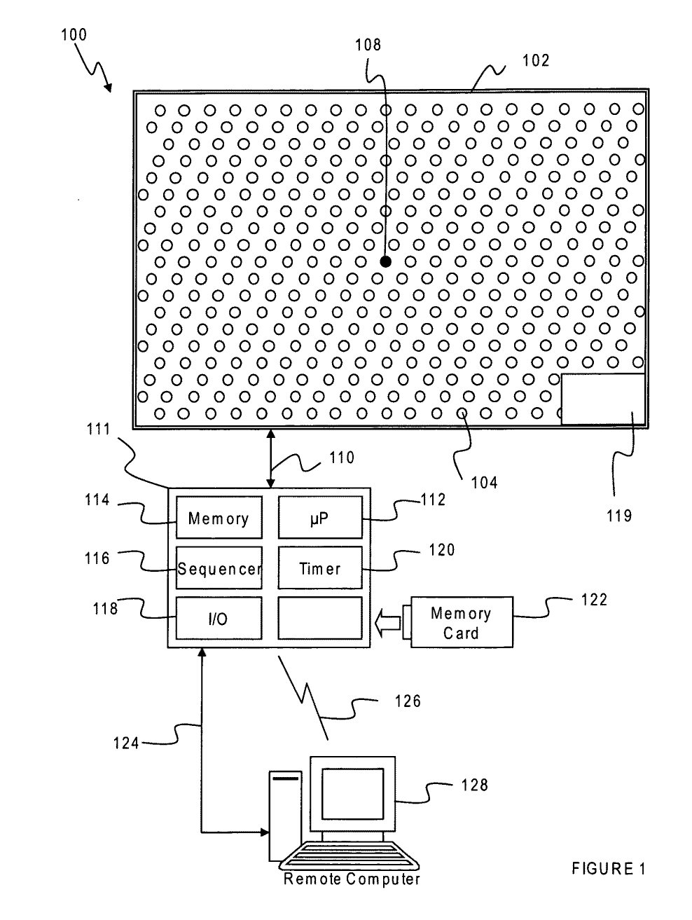

Referring initially to FIG. 1, the interactive vision therapy board of the present invention is shown and generally designated 100. Device 100 includes a display panel 102 having a number lights 104 spaced apart on the panel 102, and a fixator light 108 positioned centrally on panel 102. Panel 102 is in electrical communication through link 110 to a controller 111.

Controller 111 includes a digital processing unit 112, such as a microcontroller, micro computer, field programmable gate array (FPGA), or any other device known in the art and capable of accomplishing the functions described herein.

Also within controller 111, a memory 114, sequencer and timer 120 provide support for digital processing unit 112. For instance, memory 114 may include random access memory (RAM), program and storage read only memory (ROM), and may include a removable memory card 122, such as a USB memory stick. These memory components alone, or in combination, provide storage for program memory, historical data, and look-up data such as for specific pattern or therapy treatment protocols.

Input/Output device 118 facilitates the communication of controller 111 with the user, as well as with a remote computer 128 through wired link 124 or wireless link 126. It is to be appreciated that the present invention includes communication capabilities known in the art, and operational software may downloading into the controller 111 from the remote computer 128, and patient or other data may be uploaded from the controller 111 to the remote computer for archival or analysis purposes.

A control panel 119 may be integrated into panel 102, and provide the vision therapist, or user, to set up and control the operations of the system 100. For instance, the user may use control panel 119 to set up options for the specific therapy chosen.

The software or program could be used on a wider scale for interactive games (WI, X-box etc.) The memory card may be inserted into a USB port in which future “downloads” of new programs may be installed. New software could also be implemented, such as a hand held device to “turn off” lights for those who may be unable to “touch” the screen (i.e. patients with severe disabilities).

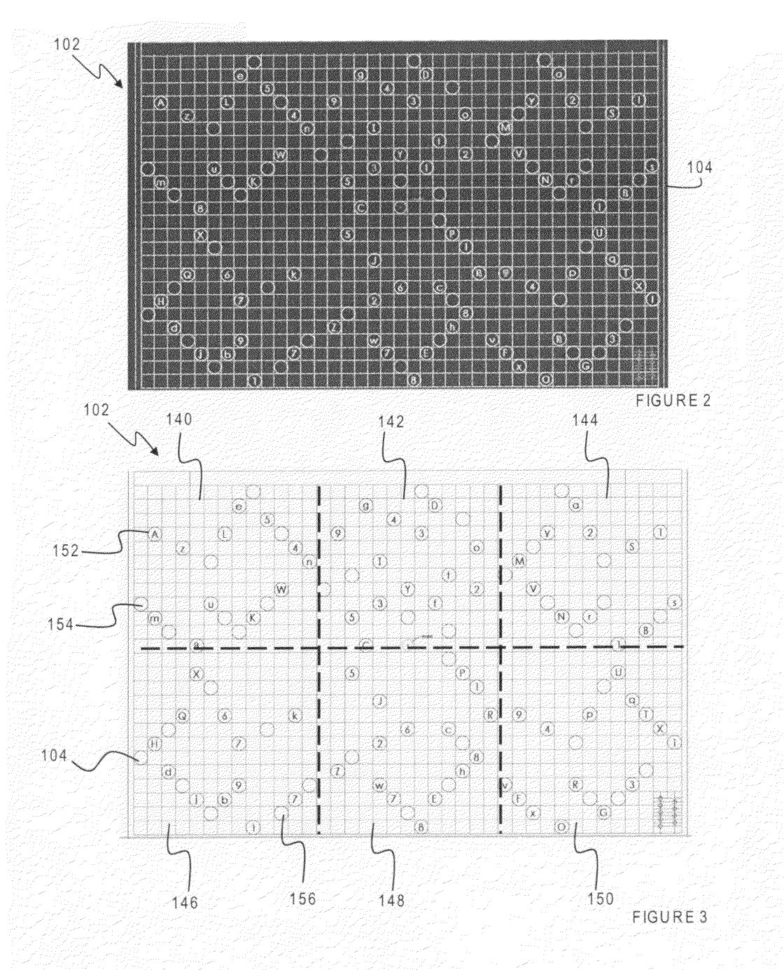

Referring now to FIGS. 2 and 3, a front plan view of the display panel 102 is shown. First, FIG. 2 shows the black background for panel 102, and the placement of lights 104 around the surface. FIG. 3 is a black and white image showing the location of lights 104, and the designation of zones.

Specifically, panel 102 includes zones 140, 142, 144, 146, 148 and 150. It is to be appreciated that panel 102 is separated into six zones, but any number of zones could be implemented in the present invention, and those zones could have shapes other than those rectangular shapes shown here.

From FIG. 3, the various types of lights can be seen. Specifically, lights 104 can include simple lights 154 (no letters or numbers), letter lights 152, and number lights 156. These lights, as will be discussed in greater detail below, can be used for specific visual training exercises.

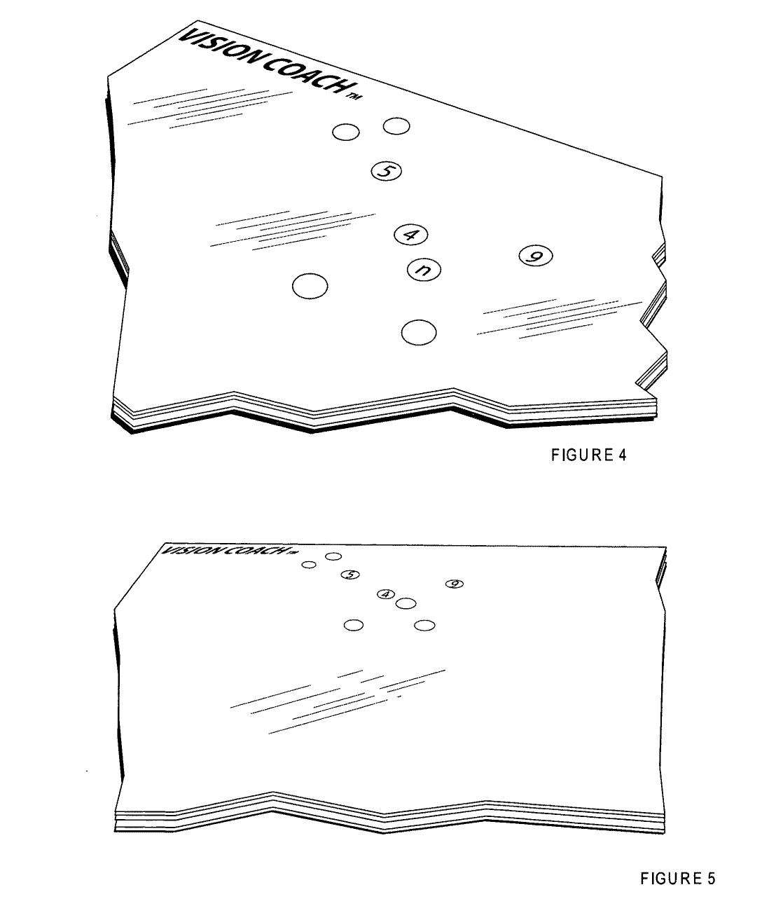

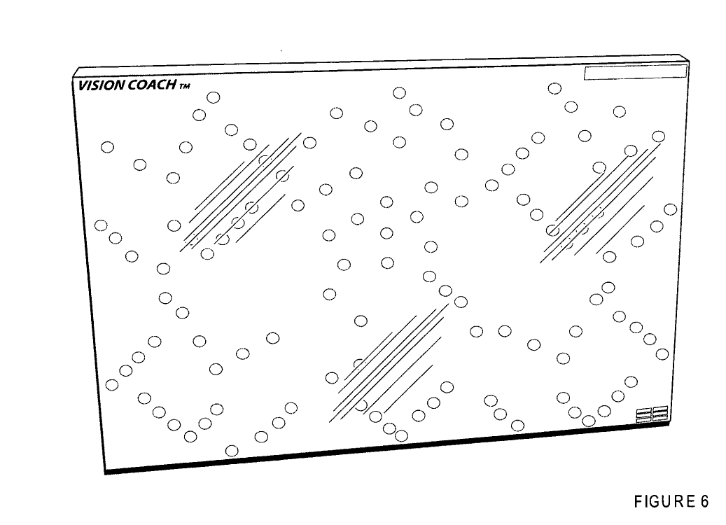

FIGS. 4 and 5 are actual photographic examples of the panel 102 of the present invention showing several lights simultaneously illuminated. FIG. 6 is a photographic example of the display panel 102 of the present invention showing the placement of the lights across the panel. FIG. 7 is a graphical representation of the placement of lights having letters, numbers, and neither letters or numbers.

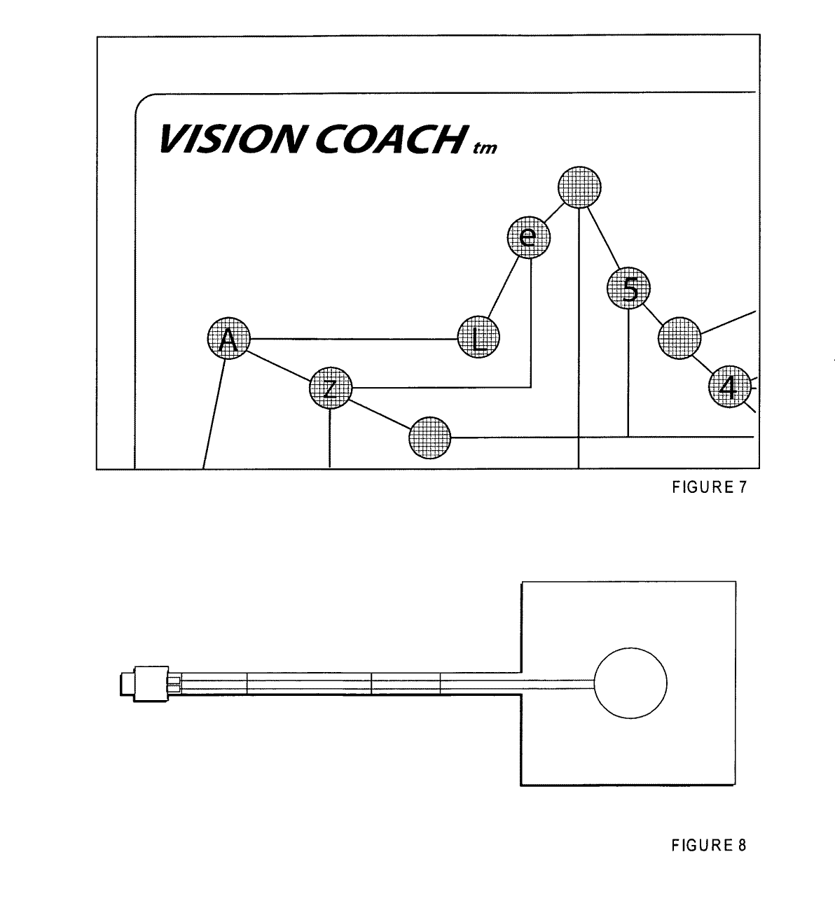

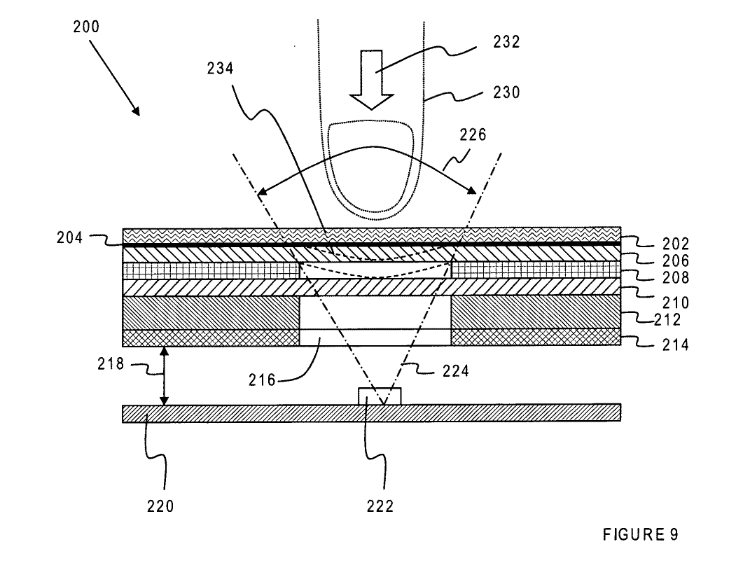

Referring to FIG. 8, an exemplary contact switch is shown. This switch is typical of the contact switch that is adjacent the front of panel 102 to detect the depression, or contact, of the light by the patient during therapy or training exercises. As will be shown in greater detail below, the switch is directly between the patient and the light, so any contact on the light surface is detected by the switch.

FIG. 9 depicts an exemplary cross-section of the panel 102 of the present invention. Specifically, cross-section 200 includes a graphic overlay sheet 202 upon which a graphic image 204 or coloring is printed on the lower side. The lower side of overlay 202 is against an upper switch contact 206 having a spacer 208 between lower switch contact 210. A support panel 212 provides rigidity to the assembly. A “honeycomb” spacer 214 is provided adjacent panel 212.

As can be seen from this figure, spacer 208, panel 212 and spacer 214 are each formed with an aperture 216 corresponding to light 104 on panel 102. Any number or letter or image can be printed on image layer 204 on overlay sheet 202. For instance, the numbers or letter can be printed, as well as shapes or animal outlines for use with younger patients.

A printed circuit board 220 is positioned a distance 218 behind spacer 214, and has a light source 222, such as a light emitting diode (LED) to provide illumination behind overlay 202. Each illumination source 222 may be manufactured to have a dispersion angle, or viewing angle, 226. In a preferred embodiment, the viewing angle of the present invention is 130 degrees. Illumination sources 222 are each equipped with red, green and white light sources. Thus, by activating the red, green, or white, or multiple lights in combination, different light sources can be generated.

It is to be appreciated that switch contacts 206 and 210 are substantially translucent thereby providing the light from light source 222 to pass through the switch and out through overlay 202 for perception by the patient. Also appreciated from this figure is that due to the sealed nature of the assembly, when no illumination source 222 is activated, the entire upper surface of the overlay appears dark as no light from any other source than sources 222 is present within the device.

When an illumination source 222 is activated, a user may push on the source to advance to the next step of the therapy. For instance, a user (depicted by finger 230) may be pushed in direction 232 to contact the surface of the overlay 202 which depresses as shown by dashed lines 234. The depressed overlay 202 in turn presses on upper switch contact 206 which deflects through spacer 208 to contact lower switch contact 210. When finger 230 is removed, the upper switch contact 206 and overlay 202 return to their original position.

The electro-mechanical switch assembly discussed herein provides a tactile feel to the user, and this results in better therapy results than a touch screen device would because the right amount of pressure must be exerted by the patient. A touch screen version would work well, however, it has little to no pressure for the patient to turn off lights as they appear on the board, and may not provide a sufficient eye-hand coordination therapy without the tactile feedback and need for applying force to turn the light off.

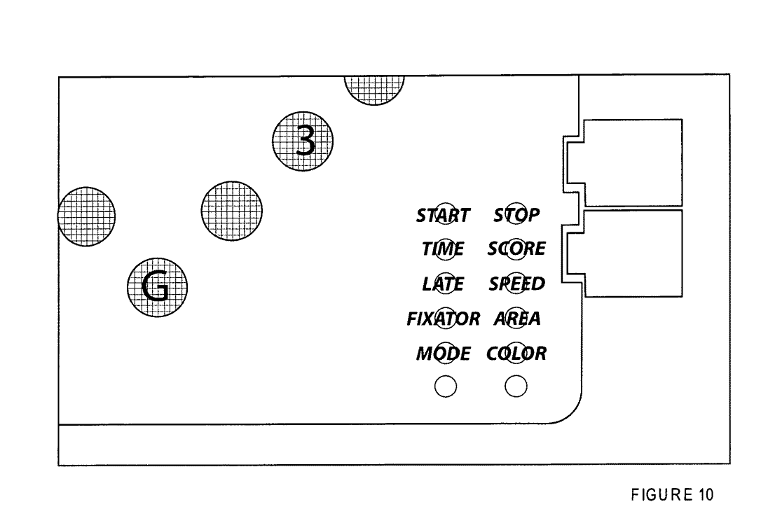

FIG. 10 is an example of the control panel 119. From this figure, some of the various features of the present invention can be seen. For instance, several user interface buttons are shown, including START, STOP, TIME, SCORE, LATE, SPEED, FIXATOR, AREA, MODE and COLOR. Other buttons and functions can be implemented without departing from the present invention.

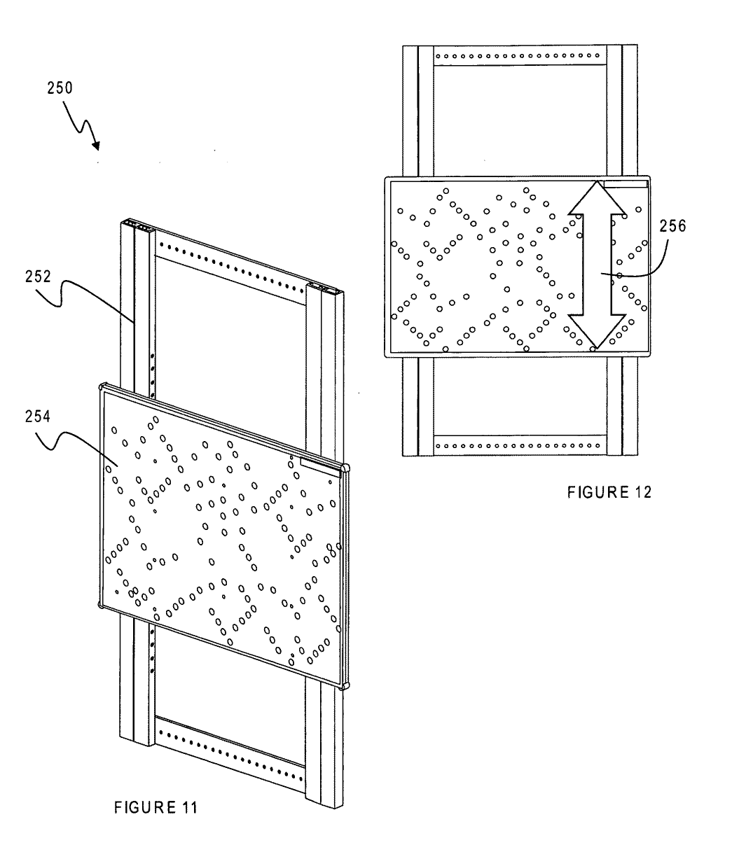

Referring now to FIGS. 11 and 12, a rack mounting for the Interactive Vision Therapy Board of the present invention is shown and generally designated 200. Rack 200 includes a pair of vertical supports 252 which support panel frame 254. As shown in FIG. 12, panel frame 254 may move vertically in direction 256 in order to match the height of the panel with the height of the patient. This allows the same Interactive Vision Therapy Board of the present invention to be used by patients of all sizes simply by raising or lowering the panel to match their height.

This devise has a mounting system that allows the therapist to slide it up and down the wall so patients that are from the range of 3′ to 6′-6″ can use it without having to remove the board from the wall and remount it. In short, the panel frame may be adjusted from a lowered position with the lower edge of the panel adjacent the floor, and a raised position with the upper edge of the panel adjacent the ceiling. This allows for the positioning of the panel to virtually any height to accommodate patients of different heights, as well as those having special needs, such as those patients in wheelchairs or unable to stand during the vision therapy sessions.

In a preferred embodiment, the mounting system allows the board to be adjusted from heights of 6′6 to 3′ and will be easily adjusted with one hand by the therapist. This board will not come off the wall once it is installed, and the corners of the board will be round to prevent sharp edges (injury).

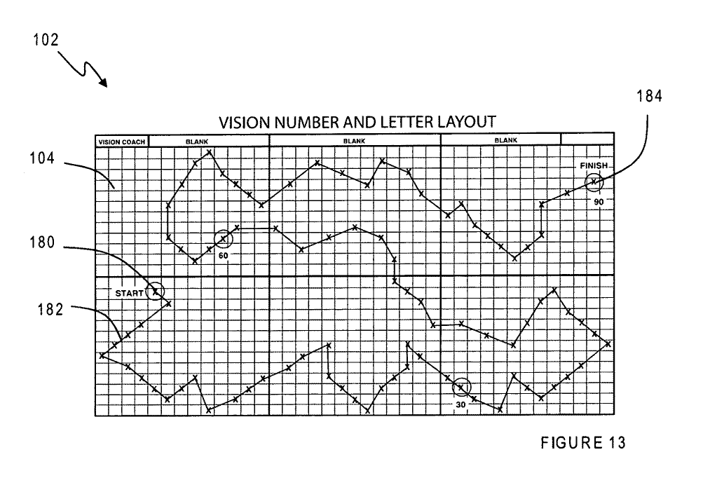

FIG. 13 depicts a sequential pattern of lights across panel 102. For instance, lights 104 may be illuminated in a sequence starting at 180, and propagating across panel 102 in directions 182 to end 184. It is to be understood that this pattern is merely exemplary of a preferred embodiment and not to be considered limiting by the path, number of light steps, etc. It is also to be appreciated that color variations, timing delays and intervals, and letter and number combinations along this sequence are fully contemplated.

### Methods of the Interactive Vision Therapy Board of the Present Invention

Because 80% of the information fed to your muscular system comes from your eyes, strengthening your visual skills is crucial to developing proficiency in sports. The skills needed in sports—dynamic visual acuity, visual concentration, peripheral awareness, tracking, focusing, visual reaction time, visual memory, visualization, and eye-hand-body coordination—can all be improved through consistent use of the Vision Coach. The goal of peak visual efficiency can be both acquired and maintained.

Dynamic Visual Acuity is the ability to clearly see objects while you and/or the objects are moving fast.

Visual Concentration is the ability to screen out distractions and stay focused on the ball or the target.

Peripheral Awareness enables you to see things that are happening around you rather than directly in front of you. When you are playing a sport, things are constantly happening both in front of you and around you, so it is crucial to increase your ability to see action to the side without having to turn your head.

Eye Tracking helps you maintain better balance and react to a situation more quickly by allowing you to follow objects without much head motion.

Good Focusing Skills allow you to see clearly both up close and in the distance, and also to shift your focus quickly, accurately, and efficiently from near to far or vice versa.

Visual Reaction Time is the speed with which your brain interprets and reacts to your opponent's action.

Good Visual Memory allows you to process and remember a fast-moving, complex picture of people and things.

Visualization is the skill that enables you to see yourself performing well in your “mind's eye” while your eyes are seeing and concentrating on something else, usually the ball. Researchers have found that the same areas of the brain that light up during performance also do so when you visualize the performance.

Eye-Hand-Body Coordination is how your hands, feet, body and other muscles respond to the information gathered through your eyes. It is an important part of most sports because it affects both timing and body control.

Depth Perception enables you to quickly and accurately judge the distance between yourself, the ball, your opponents, teammates, boundary lines and other objects.

The ability to combine red, green, white lights is highly innovative. Red and green combine light to make black, which indeed cannot be distinguished from a black overlay—no perceptible light. Utilizing this phenomenon, the patient's ability to utilize each eye equally is determined, and based on this determination, particular therapies can be developed.

In one case, the use of red/green for stereopsis (binocularity) has been developed by the present inventor, an Optometric Vision therapist with more than ten years experience working with special populations. In fact, this inventor has established multiple uses for a diversity of patients. Primarily with the use of red and green lights this invention will be used to help “train” the brain (eye/s) to become binocular. This will in turn enhance or develop depth perception and binocularity (the use of both eyes/teaming).

By utilization of the Interactive Vision Therapy Board of the present invention, treatment and therapy for each of these skills can be realized. As can be appreciated, the present invention provides a tool to experienced vision therapists for the diagnosis, treatment and individualized therapy to improve their patient's vision related health. This disclosure includes a few exemplars of typical treatment modalities and therapies which can be realized using the Interactive Vision Therapy Board of the present invention.

The Interactive Vision Therapy Board of the present invention includes a red light, green light, and in some applications, a white light. In order to understand the benefits of having a red and green light within the Interactive Vision Therapy Board of the present invention, it is important to understand how red/green glasses assist with the diagnosis and treatment of vision issues. When using the description of the red/green glasses the Optometric correct term would be red green anaglyphic glasses.

To make it as easy as possible to understand, this discussion refers to a patient's right eye as the “red” eye. Typical red/green glasses will have red on the right and green on the left, and some are even made to be switched out but we will use red-right, green-left. When using the R/G glasses the background you choose to use will give you a different effect. For instance, the Interactive Vision Therapy Board of the present invention has a black overlay on the board or panel.

With a red light up on the board, with R/G glasses on the patient should be able to see the red light if both eyes are working together as they should be. Now, If the red eye is covered, the patient should not be able to see anything. Everything should be black.

In order to understand why the patient could see nothing, it is important to note that red and green combine together to make black. If the patient uncovers the red eye, he should be able to see the red light again. When the patient covers his green eye, the patient should see the red light. This is because the red eye “sees” red and green eye “sees” green.

In patients afflicted with an eye turn (one eye is pointing straight and one eye is turned in) the eye that is pointing straight is the ONLY eye that is taking information into the brain. The eye that is turned is “suppressing” information. If the brain is not suppressing information from the turned eye then the patient will see double as one eye will be pointing straight at an object and the turned eye will be pointing at something else. Most likely the brain chooses to suppress. This results in loss of depth perception (3-D) and loss of information to the brain.

With the use of R/G on the Interactive Vision Therapy Board of the present invention that patient can “train” the brain (eyes) to “see” what they should see.

Some therapies can utilize the fixator light in the middle of the board (which cannot be green or red). The fixator light needs to be white (or white like, such as silver), so that with R/G glasses on the center light (white) should be what is called in the Vision Therapy world, “luminescent” or “luster”. This is a “blend” of red and green. If the patient did not see “luster”, yet responded that the enter light was RED, it would indicate that the patient was only “seeing” with their RED eye (green eye suppressing). If the response was GREEN, it would be the green eye “seeing” (red eye suppressing). When looking at a white object with R/G on, RED eye “sees” red and GREEN eye “sees” green. Both eyes together will (should) see “luster”.

### Operational Examples of the Interactive Vision Therapy Board

### Random Sequence—Time Trials

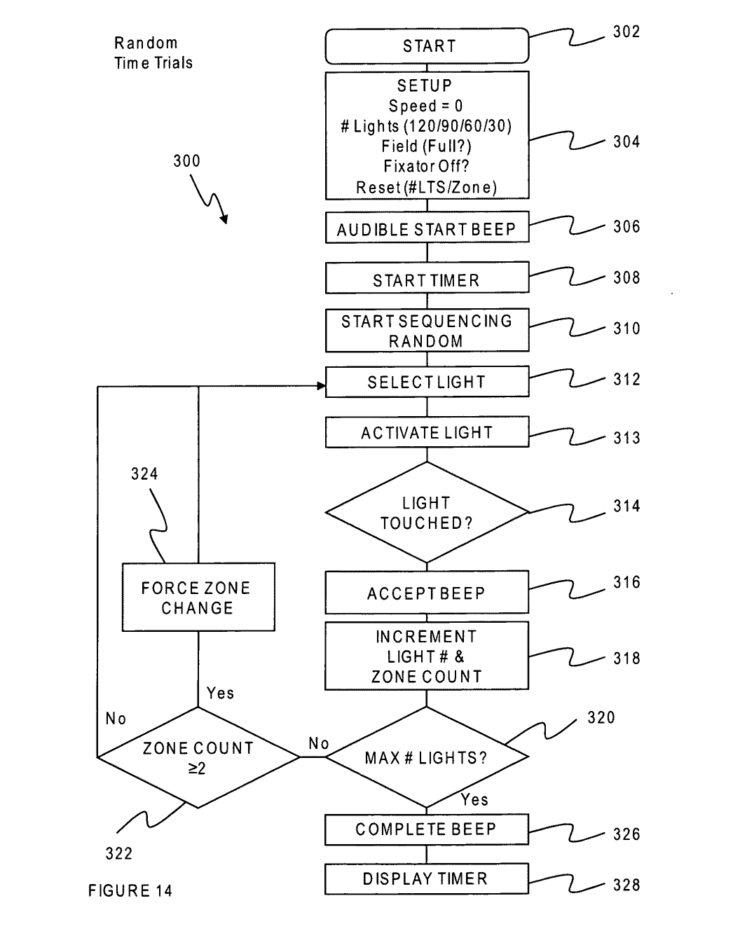

Referring now to FIG. 14, a flow chart diagram showing an exemplary operation of the Interactive Vision Therapy Board of the present invention is shown and generally designated 300. This method is known as a Random/Time Trials method. Method 300 begins with step 302 and proceeds through a setup step 304. During this setup step 304, the speed is set to zero indicating no delay is incorporated into the light sequence. The number of lights are selected from a set of 120, 90, 60, or 30. Other numbers of lights can be used. The fixator light is set to OFF, and the zone and light counters are reset.

Step 306 includes an audible start beep to indicate to the patient that the sequence is starting. The sequence starts the timer in step 308 and begins creating a random light sequence in step 310. The light to illuminate is selected in step 312 and the light is turned on in step 313. The method waits until the light is touched in step 314, and once the switch is pressed, an audible confirmation beep is sounded in step 316. The light number and zone counters are incremented in step 318.

The present invention maintains counts of the number of lights that have been in that sequence, and the number of times a light has been in the same zone. It is important in some applications to avoid too many lights in the same zone, such as in a full-field test. This limit may be set by the user, and if the limit is exceeded, the method forces a change in the zone.

If the number of lights for the sequence has not been reached in step 320, the method advances to step 322 where the zone count is checked. If there has been more than the number of allowable lights in a zone, the zone is forced to change in step 324. The method then selects another light in step 312 and the process repeats.

If the maximum number of lights in the sequence was reached in step 320, a completion beep is sounded in step 326, and the timer is displayed in step 328. By keeping track of the patient's display timer scores, the improvement of the patient's eye hand coordination may be monitored.

The present method improves central and peripheral awareness (sports, reading, driving . . . daily living skills), visually guided movement, eye-hand coordination, reaction time (speed), large eye movements. All or any of the above could be used for a variety of patients ranging from Cerebral Palsy patients with reduced mobility, TBI or ABI patients with midline issues to high achieving sports athletes wanting to improve or enhance the skills they already have. This is particularly useful since peripheral vision/awareness is responsible for balance. Without balance our bodies are challenged with day to day activities.

### Random Sequence—Time Trials—Color

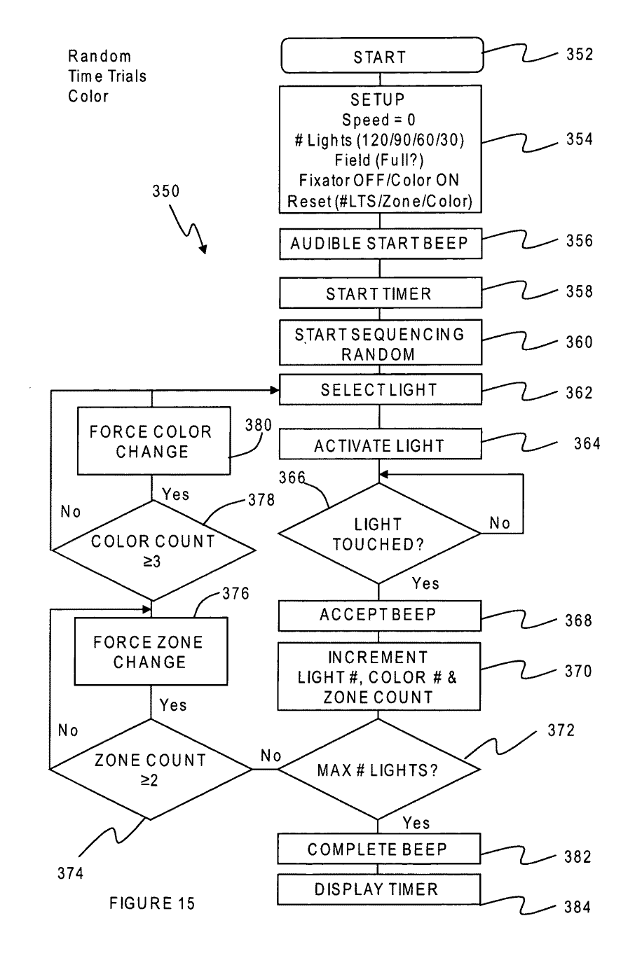

Referring now to FIG. 15, a flow chart diagram showing an exemplary operation of the Interactive Vision Therapy Board of the present invention is shown and generally designated 350. This method is known as a Random/Time Trials/Color method. Method 350 begins with step 352 and proceeds through a setup step 354. During this setup step 354, the speed is set to zero indicating no delay is incorporated into the light sequence. The number of lights are selected from a set of 120, 90, 60, or 30. Other numbers of lights can be used. The fixator light is set to OFF, and the zone, light and color counters are reset.

Step 356 includes an audible start beep to indicate to the patient that the sequence is starting. The sequence starts the timer in step 358 and begins creating a random light sequence in step 360. The light to illuminate is selected in step 362 and the light is turned on in step 364. The method waits until the light is touched in step 366, and once the switch is pressed, an audible confirmation beep is sounded in step 368. The light number, color, and zone counters are incremented in step 370.

If the number of lights for the sequence has not been reached in step 372, the method advances to step 374 where the zone count is checked. If there has been more than the number of allowable lights in a zone, the zone is forced to change in step 376. Next, the color count is checked in step 378, and if the maximum number of lights having the same color has occurred, a color change is forced in step 380. The method then selects another light in step 362 and the process repeats.

If the maximum number of lights in the sequence was reached in step 372, a completion been is sounded in step 382, and the timer is displayed in step 384.

All of the above mentioned conditions with the added treatment, training and enhancement of binocularity due to the usage of the Red and green lights. Any reduced number other than the last mentioned would be used possibly for younger children or CP, TBI, ABI, Autistic populations, etc as attention to the task may be limited.

### Sequential—Time Delay

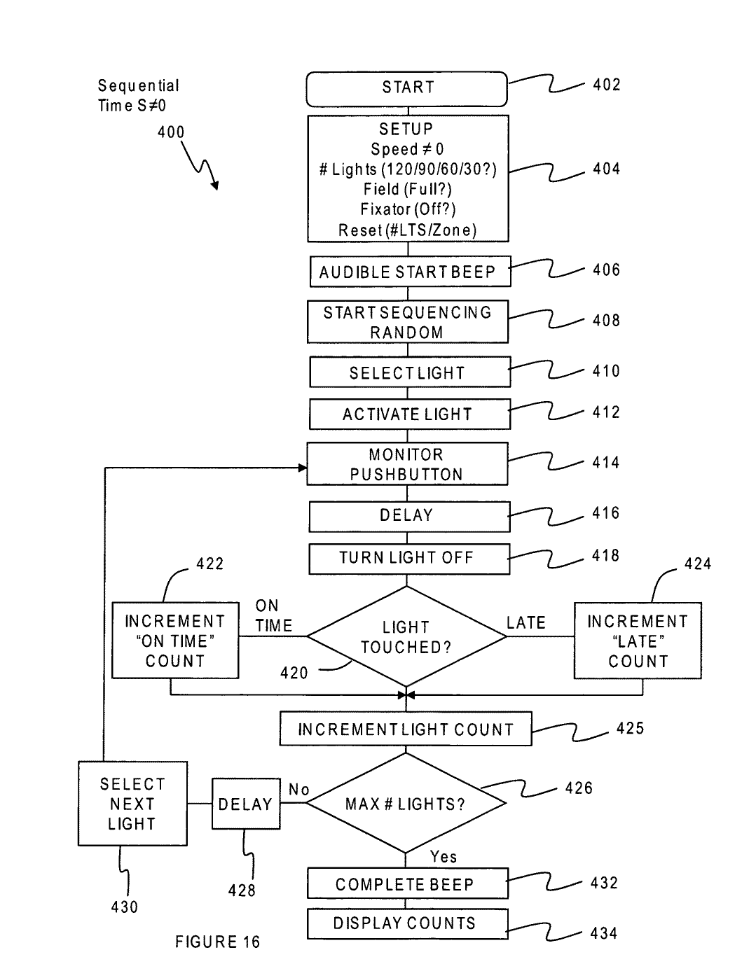

Referring now to FIG. 16, a flow chart diagram showing an exemplary operation of the Interactive Vision Therapy Board of the present invention is shown and generally designated 400. This method is known as a Sequential/Time Delay method. Method 400 begins with step 402 and proceeds through a setup step 404. During this setup step 404, the speed is set to a non-zero value indicating that a delay is incorporated into the light sequence. In a preferred embodiment, the speeds available are as follows:

Speed 1=2000 ms/light=0.500 lights per second
Speed 2=1333 ms/light=0.750 lights per second
Speed 3=1000 ms/light=1.000 lights per second
Speed 4=667 ms/light=1.500 lights per second
Speed 5=500 ms/light=2.000 lights per second
Speed 6=444 ms/light=2.250 lights per second
Speed 7=400 ms/light=2.500 lights per second
Speed 8=364 ms/light=2.750 lights per second
Speed 9=308 ms/light=3.250 lights per second
Speed 10=267 ms/light=3.750 lights per second
Speed 11=211 ms/light=4.750 lights per second

The number of lights is selected from a set of 120, 90, 60, or 30. Other numbers of lights can be used. The fixator light is set to OFF, and the zone and light counters are reset.

Step 406 includes an audible start beep to indicate to the patient that the sequence is starting. A random light sequence is created in step 408. The light to illuminate is selected in step 410 and the light is turned on in step 412.

The method monitors the pushbutton associated with the selected light in step 414 and a delay 416 determines the duration of the light until the light is turned off in step 418. If it is determined that the pushbutton was pushed in step 420, if the patient pushed the button during the lighted period, the “on time” count is incremented in step 422. However, if the pushbutton was pushed after the lighted period, the “late” count is incremented in step 424. The light number is incremented in step 425.

If the number of lights for the sequence has not been reached in step 426, the method advances to a delay step 428, the next light in the sequence is selected in step 430 and the method repeats at step 414. If the maximum number of lights in the sequence was reached in step 426, a completion beep is sounded in step 432, and the “on time” and “late” counts are displayed in step 434.

This method is particularly useful for sports training, Police/Pilot training for awareness of surroundings/safety, and improves reaction time, eye hand coordination, speed training, R/G binocularity enhancement.

### Fixator—Active—Time Delay

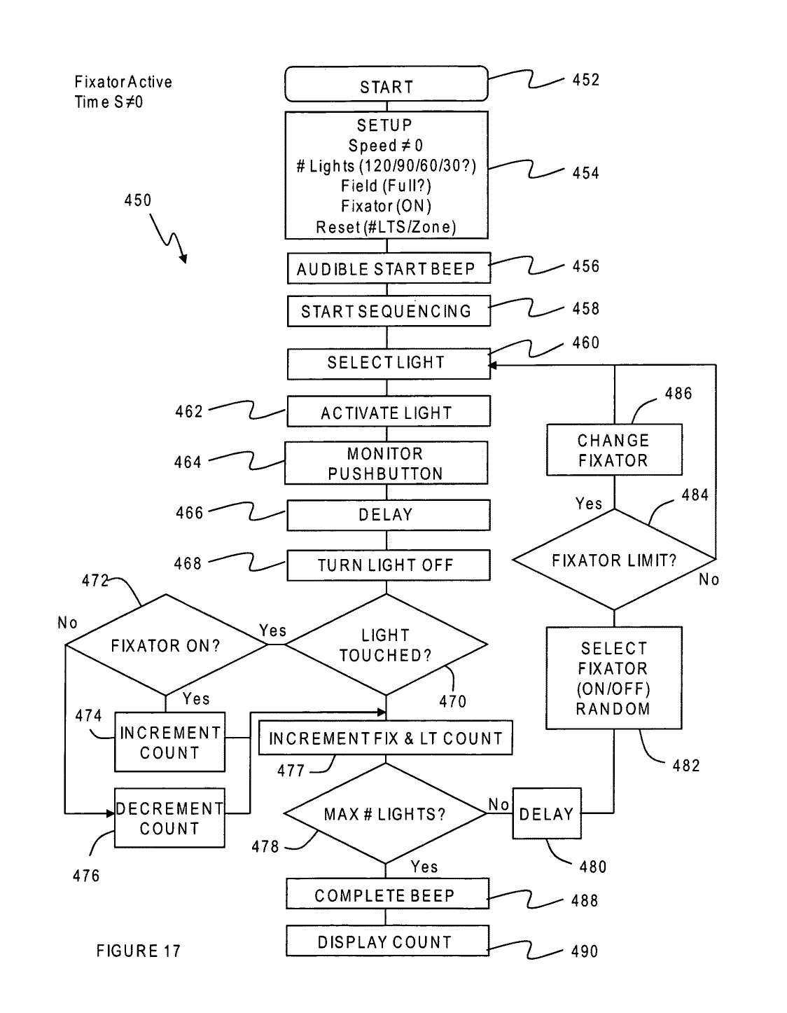

Referring now to FIG. 17, a flow chart diagram showing an exemplary operation of the Interactive Vision Therapy Board of the present invention is shown and generally designated 450. This method is known as a fixator/Active/Time Delay method. Method 450 begins with step 452 and proceeds through a setup step 454. During this setup step 454, the speed is set to provide for a delay to be incorporated into the light sequence. The number of lights is selected from a set of 120, 90, 60, or 30. The field is selected as “FULL” or partial, and the fixator light is set to ON

Step 456 includes an audible start beep to indicate to the patient that the sequence is starting. The sequence starts in step 458 and begins creating a random light sequence. The light to illuminate is selected in step 460 and the light is turned on in step 462.

The method monitors the pushbutton associated with the selected light in step 464 and a delay 466 determines the duration of the light until the light is turned off in step 468. If it is determined that the pushbutton was pushed in step 470, the method then determines if if the patient pushed the button when the fixator light was ON in step 472. If the fixator light was ON, the counter is incremented in step 474, however, if the fixator light was OFF, then the patient should not have pushed the button associated with the light, and the counter is decremented in step 476. The light number is incremented in step 477.

If the number of lights for the sequence has not been reached in step 477, the method advances to a delay step 480, and the fixator state is randomly chosen in step 482. It is advantageous to avoid having the fixator light either on or off for extended periods of time, so step 484 determines whether the fixator state limit has been reached, and if so, the fixator state is forced to change in step 486. The method selects another light in step 460 and the method repeats.

If the maximum number of lights in the sequence was reached in step 478, a completion been is sounded in step 488, and the counter is displayed in step 490.

The use of the fixator active would be used to enhance awareness of central and peripheral vision, cognitive reaction time/speed, endurance, ability to choose to react or not to react to a situation.

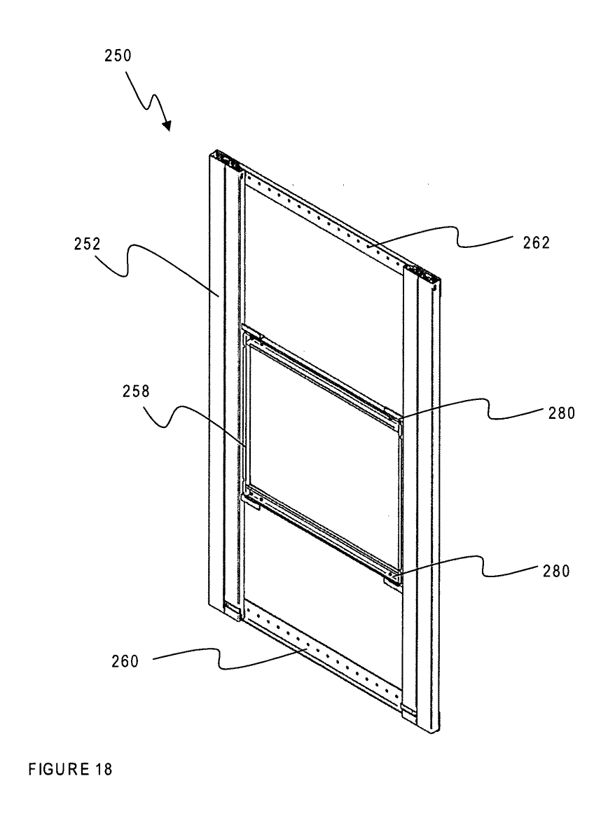

Referring now to FIGS. 18-22, exemplary operations manual for the Interactive Vision Therapy Board of the present invention are shown. While the operations manual and the methods set forth above are indicative of the operation of a preferred embodiment, it is to be appreciated that these are merely exemplars of the operation of the present invention, and the particular method sequences or methods of operation are not to be considered limiting. Rather, the scope of the present invention is to be as broad as disclosed herein, and not limited to a particular application, therapy or treatment described.

It is also to be appreciated that there are several guidelines which, when combined with the present invention, will prove beneficial for the diagnosis and treatment of visual disorders. For instance, any reduced field would be used to possibly train young children, TBI, ABI, or for PT (for example) for central/peripheral vision, visually guided movement, crossing the midline, the use of both hands/eyes/both sides of the body, reduced mobility as with partial paralysis. Any higher number of the reduced field would be used as a “higher” level for challenging. With the addition of the red and green with ANY mode you would then be introducing the enhancement of binocularity.

A TBI that might suffer from a homonomous hemianopsia for instance would use one of the upper or lower fields(example of fields) to work on developing skilled vision of the “intact” field and making large eye movements into the “non” intact field and to judge spatial location of visual targets in the “non” intact field. Sports training would help the athlete to be aware of all of the visual field and to pin point a field that might have a deficit.

The sequential lights (pattern) could be use for speech and language for articulation and recognition of letters and numbers. At speed “0” it could be used for training those that suffer from stuttering. An autistic child is able to direct his/her attention to the light and accurately predict the appearance of the next light. The sequential pattern could also be use to work on saccadic eye movement and along with color that would enhance binocularity and efficient eye teaming that provides accuracy as to where the eyes are pointing.

An exemplary Operational Manual is included in the following pages, and is intended to describe a preferred embodiment of the present invention. It is to be noted, however, that the sequence and descriptions outlined in the Operations Manual is merely exemplary of a preferred embodiment, and is intended to exemplify the versatility of the present invention in the treatment of vision therapies.

### Vision Coach Operating Manual

### Power Switch

The power switch is located on the lower right side of the Vision Coach. Flip the switch to turn the power on and off.

The Vision Coach will run a quick self test to ensure all LED's are functioning. When working correctly the lights will flash on and off down the board and three quick tones will sound. This signals you are ready to begin. The alphanumeric LED's are located in the upper right hand corner. Time, score, etc. will be kept track in this area.

When powered on, the default setting will be:

### Full Field

120 lights/no emphasis

### Random Pattern

### Fixator OFF

### Speed 0

### Color RED

The key pad on the lower right side may be used at any time to change the default settings.

### Start

Pressing the start button will activate the Vision Coach. You will hear three quick “stand by” tones followed by a “go” tone. This gives two seconds to prepare to start. The exercise will carry on with the most recently programmed settings and will continue on until the appropriate exercise has been completed or stopped. During the exercise the LED display will show the speed on the left and the score on the right. To keep the LED area blank during an exercise, press the start button twice rapidly. The LED information will appear when the exercise is completed or stopped.

### Stop

Press the stop button once to terminate an exercise before completion.

### Time

Press the time button to display the number of seconds elapsed during the exercise. The display will exhibit: TIME followed by seconds.

### Example: TIME 65

### Score

Press the score button to display the number of hits from the last exercise. The display will exhibit: SCORE followed by seconds. Example: SCORE 45

### Late

Press the late button to display the number of late hits that occurred with the last exercise. The display will exhibit: LATE followed by the number of inaccurate hits. Example: LATE 32

### Fixator

Pressing the fixator button will show the current setting. Consecutive presses will display the following program options:

FIX ON—Fixator light stays on throughout exercise.
FIX OFF—Fixator light stays off for exercise.
FIX ACTIVE—Fixator light will come on and off randomly throughout exercise. If a light is hit while the fixator light is not on you will hear a double tone to indicate an inaccuracy.

### Active Fixator Instructions

When set on active the fixator will turn on and off randomly during the exercise. It will always be “on” at the start of the exercise. The fixator may remain lit for one to fifteen hits and can remain unlit for one to five hits.

### Area

Press the area button to show the current area setting. Consecutive presses will cycle through the following options allowing emphasis in a particular area of the visual field. The lights will display in the area of the board chosen. The following sections of the board may be selected for use.

No Emphasis: The board is used evenly (default). The display will exhibit: NO EMPH

Upper Left: Lights will randomly be displayed in the upper left field. The display will exhibit: UP LFT

Lower Left: Lights will randomly be displayed in the lower left field. The display will exhibit: LOW LFT

Left Side: Lights will randomly be displayed on the left side of the board. The display will exhibit: LFT SIDE

Upper Right: Lights will randomly be displayed in the upper right field. The display will exhibit: UP RT

Lower Right: Lights will randomly be displayed in the lower right field. The display will exhibit: LOW RT

Right Side: Lights will randomly be displayed on the right side of the board. The display will exhibit: RT SIDE

Upper Field: Lights will randomly be displayed on the upper half field of the board. The display will exhibit: UP FLD

Lower Field: Lights will randomly be displayed on the lower half field of the board. The display will exhibit: LOW FLD

Full Field Lights entire board is active. Lights will randomly be displayed on the full field. The display will exhibit: FULL FLD

Note: When using any reduced field or sequential pattern (see below) the emphasis will not take effect.

### Mode

Press the mode button for the present mode to be displayed. Consecutive presses will run through the following options:

Reduced Field 60 will exhibit: RF 60. A reduced field of 60 random lights will be displayed. Reduced Field 80 will exhibit: RF 80. A reduced field of 80 random lights will be displayed.

Full Field 30 will exhibit: FF 30; A full field of 30 random lights will be displayed. Full Field 60 will exhibit: FF 60. A full field of 60 random lights will be displayed.

Full Field 90 will exhibit: FF 90. A full field of 90 random lights will be displayed. Full Field 120 will exhibit: FF 120. A full field of 120 random lights will be displayed.

Sequential 30 will exhibit: SEQ 30, A sequential pattern of 30 lights will be displayed. Sequential 60 will exhibit: SEQ 60, A sequential pattern of 60 lights will be displayed. Sequential 90 will exhibit: SEQ 90. A sequential pattern of 90 lights will be displayed.

### Color

The 120 LED's provide two different colors, red and green.

Press the color button to show the current color setting. Consecutive presses cycle through the color options: Red—Use for red lights only during exercise. The display will exhibit: RED. Green—Use for green lights only during exercise. The display will exhibit: GREEN. Red/Green—red and green lights will appear randomly during the exercise. The display will exhibit: RED/GREEN

Referring now to FIG. 18, a front perspective view of the mounting system of the present invention is shown and generally designated 250. Frame 250 includes a wall-mounted frame 252 with a central slide frame 258 that slides up and down within the wall mounted frame 252 and is used to secure the display panel (not shown this Figure) to the wall through attachment brackets 260 and 262 for adjustment and use. As will be discussed in greater detail below, the ability to elevate the central slide frame 258 up and down provides for the most appropriate positioning of the display panel for use.

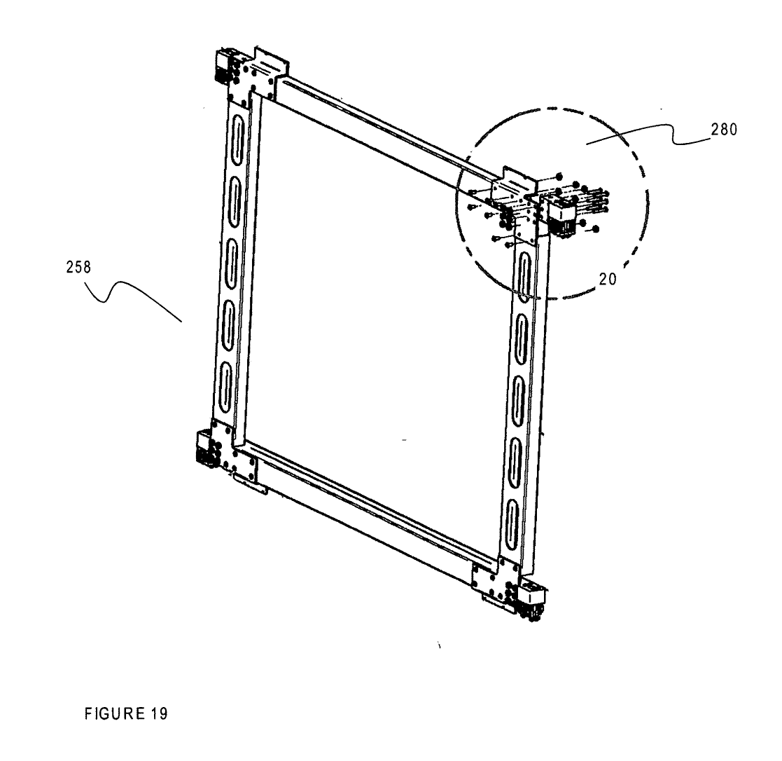

FIG. 19 is a perspective view of the central slide frame 258 showing an exploded brake assembly 280 which is used to facilitate the vertical positioning of the slide 258 within the wall mounted frame 252. As shown, central slide frame 258 includes four separate brake assemblies 280 (two on each side), which provide a stabilizing and locking mechanism for the four corners of the display panel. The ability to fix the position of the display panel during use is key in that since the visual and physical coordination is being measured, the absence of movement of the display panel provides for a more effective, and more accurate assessment, of the vision therapy treatments.

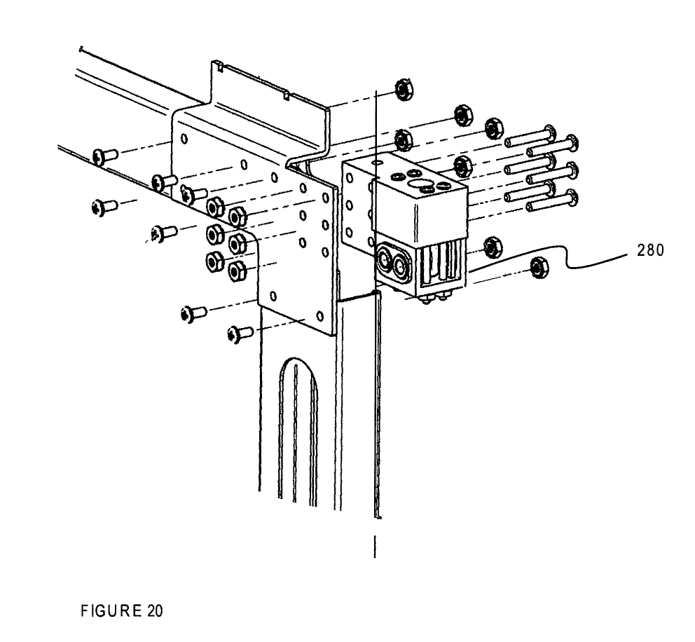

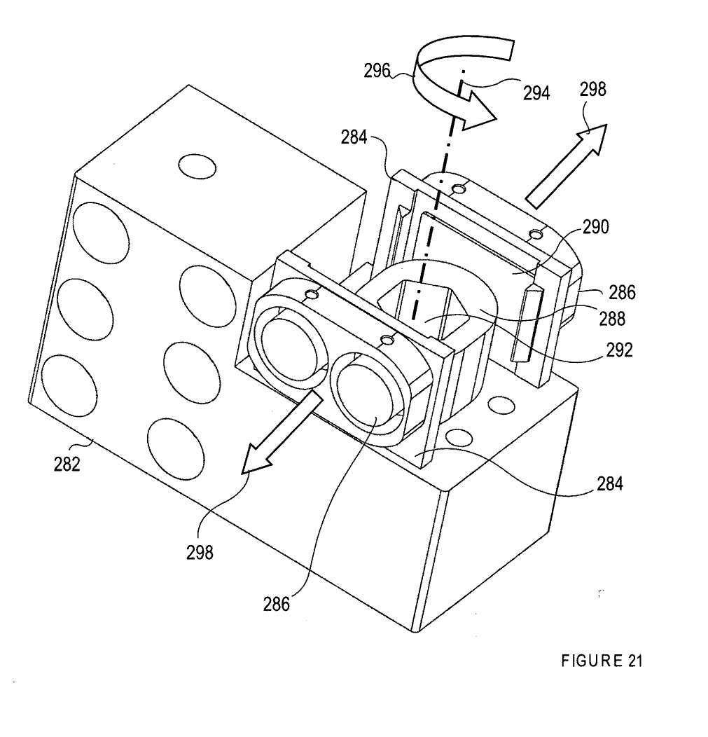

Referring to FIG. 20, an enlarged view of the brake assembly 280 as it mounts to the corner of the central slide frame 258 is shown. From this figure, it can be seen that the brake assemblies 280 are securely attached to the central frame slide 258 such that there is no chance of movement of the central frame slide 258 if the brakes within the brake assembly are locked.

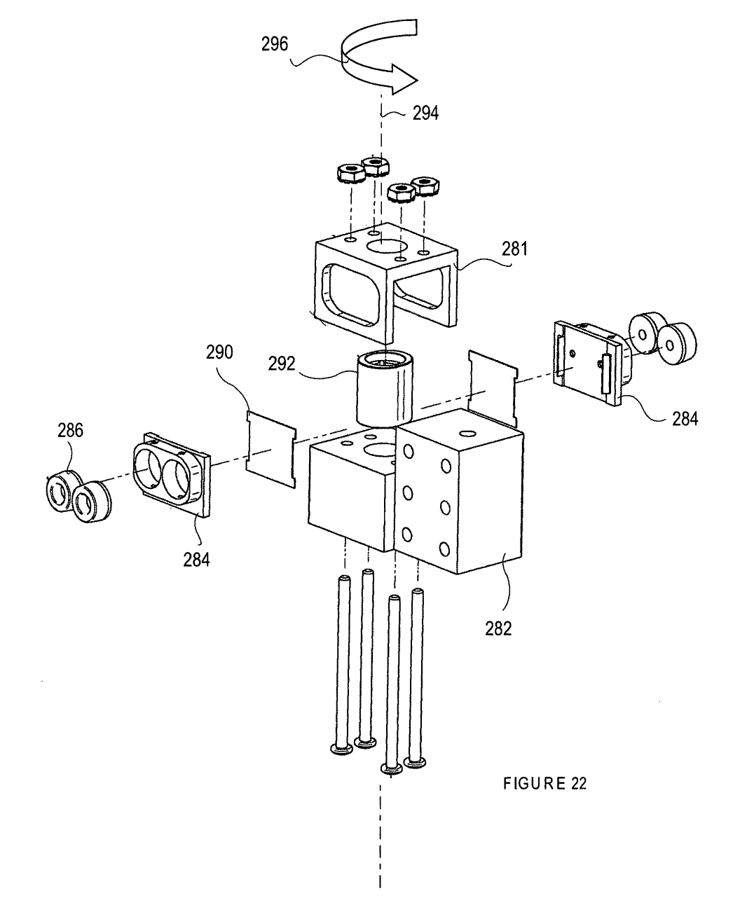

FIG. 21 shows an enlarged view of the brake assembly 280 with a top cover (281 shown in FIG. 22) removed for clarity. Brake assembly 280 includes block 282 having a pair of brake pad receivers 284 that each contain a pair of brake pads 286. A metallic spring panel 290 is adjacent and attached to each receiver 284 to provide a spring force inwards toward the center of the block where cam 292 is positioned. As shown, cam 292 is oval in shape and formed with an axial bore 294 formed with a hexagonal cross-section and rotatable in direction 296. Brake assembly is positioned within frame members that are attached to a wall and in use, the cam 292 is rotated about axis 294 to overcome spring forces provided by spring panels 290 such that brake pads 286 are urged outwards in directions 298 to contact the interior walls of the wall mounted frame 252.

Referring to FIG. 22, an exploded view of the brake assembly 280 is shown. This figure includes cover 281 as it is positioned to capture brake assembly components against block 282 and to provide structural support to the positioning of receivers 284. From this figure, it is to be appreciated that the insertion of the brake pads 286 within receivers 284 allows the pads to be forced outwards when the central cam 292 is rotated. This rotation is achieved by the rotation of a locking arm to be discussed below. Also, it should be appreciated that the brake pads 286 are either free floating within the receiver 284 when the cam 292 is rotated to push on the springs 290, or urged outwards when the cam 292 is positioned not to push on the springs 290. In that case, the brake assembly can be freely slid within the frame 258.

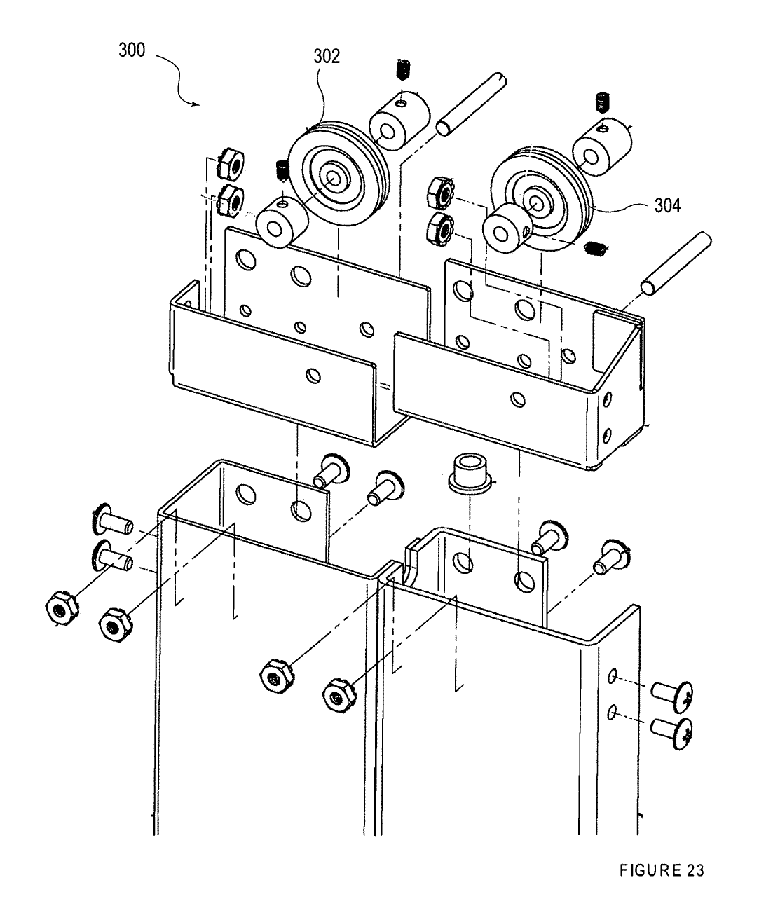

FIG. 23 is an exploded view of the pulley system 300 of the present invention showing the pair of pulleys 302 used to support the central slide frame within the wall mounted frame and balance the weight with a counterweight to facilitate the raising and lowering of the central slide frame and attached display panel.

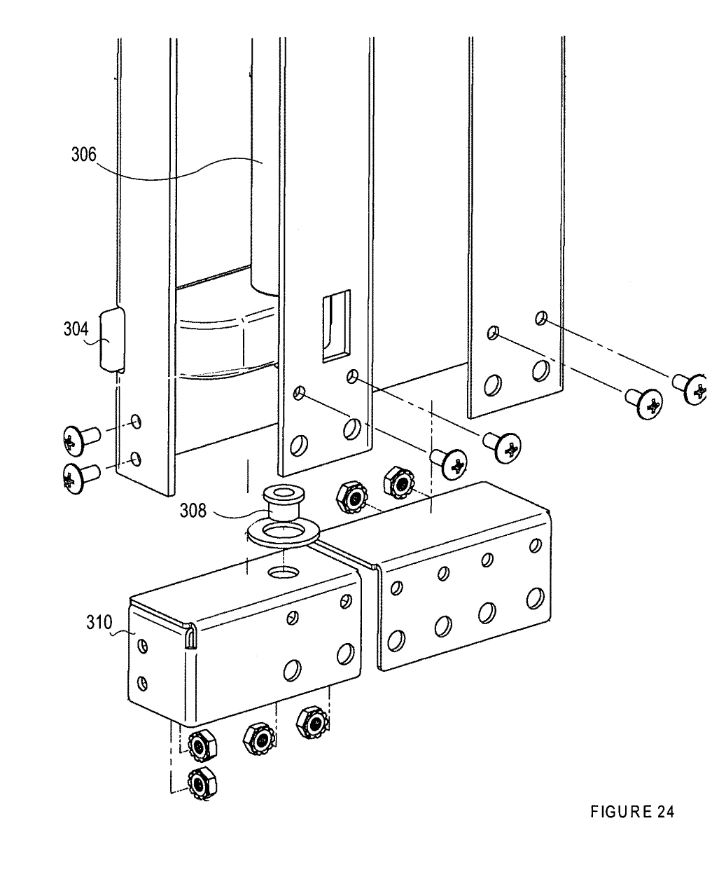

FIG. 24 is a view of the wall mounted frame and showing the locking lever 304 attached to the end of the cam rod 306 that passes through the cam within the brake assembly so that the locking lever can be rotated 90 degrees to remove the forces from brake assembly in the central slide frame, the frame can be raised and lowered, and the locking lever can be returned to its position rotating 90 degrees to rotate the cam within the brake assembly to lock the central slide frame in place. The cam rod 306 is mounted to a bearing 308 that is positioned within insert 310 and attached to frame rail 258.

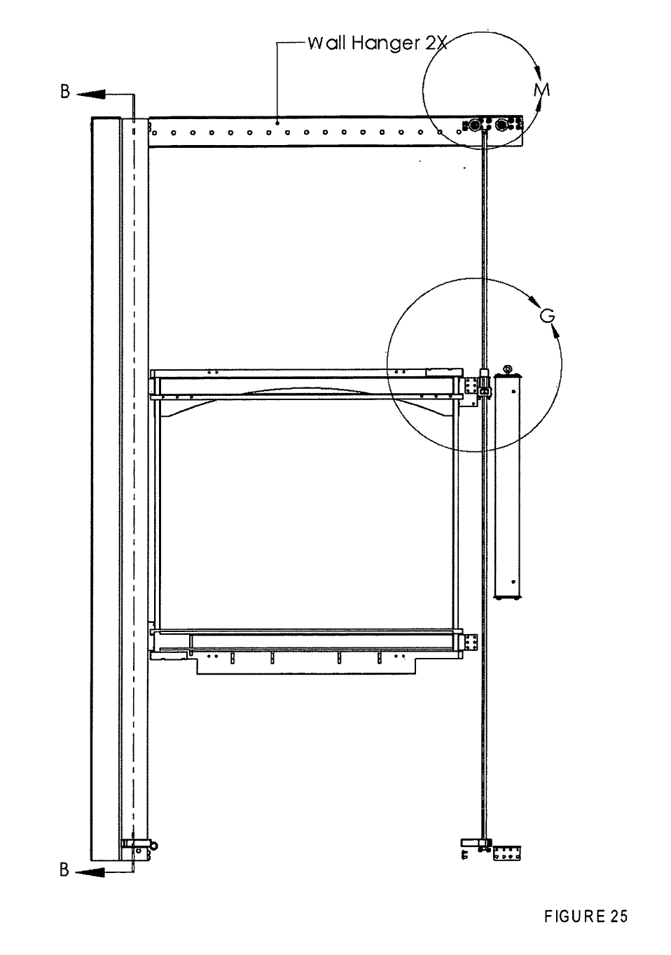

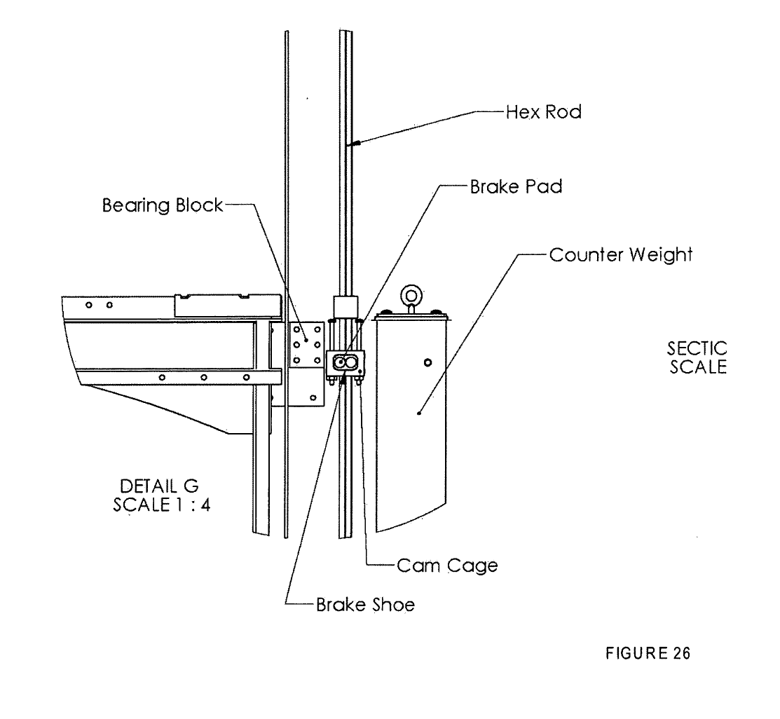

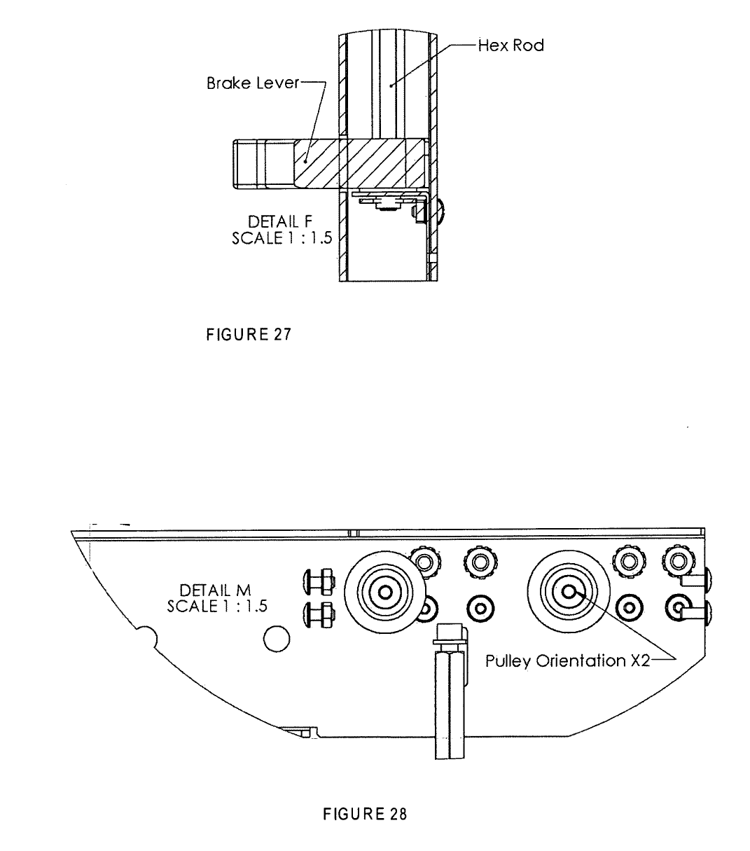

FIGS. 25, 26, 27 and 28 depict manufacturing figures for the frame and locking assembly of the present invention. As shown the counterweight is attached to the pulley system and facilitates the raising and lowering of the display panel.

While the particular Interactive Vision Therapy Board as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

1. An interactive vision therapy board, comprising:
a display panel having a plurality of selectively illuminated lights;
a plurality of finger activated switches responsive to a patient touch,
and a means for changing said light pattern in response to said switches.
